(19) United States
(12) Patent Application Publication
(10) Pub. No.: US 2020/0141949 A1
(43) Pub. Date: May 7, 2020

(54) USE OF THE RHO GDP DISSOCIATION INHIBITOR 2 PROTEIN AS A DIAGNOSTIC AND PROGNOSTIC MARKER OF INTESTINAL INFLAMMATORY DISEASES

(71) Applicants: Laura STRONATI, Rome (IT); Roberta VITALI, Rome (IT); Salvatore CUCCHIARA, Rome (IT)

(72) Inventors: Laura STRONATI, Rome (IT); Roberta VITALI, Rome (IT); Salvatore CUCCHIARA, Rome (IT)

(21) Appl. No.: 16/632,709

(22) PCT Filed: Jul. 18, 2018
(86) PCT No.: PCT/IB2018/055326

§ 371 Date: Jan. 21, 2020

(30) Foreign Application Priority Data
Jul. 20, 2017 (IT) 102017000083055

(51) Int. Cl. G01N 33/68; G01N 1/28

(52) U.S. Cl. CPC G01N 33/6893; G01N 1/286; G01N 2800/067; G01N 2001/2866; G01N 2800/065

(57) ABSTRACT

Disclosed is a method that uses Rho GDP dissociation inhibitory 2 protein as a diagnostic and prognostic marker of inflammatory bowel diseases by way of the ex vivo detection of the protein level in faecal samples of patients.

### FIELD OF APPLICATION

The present invention is directed to the diagnostic, prognostic and response utilization of the Rho GDP dissociation inhibitor 2 for detecting conditions of chronic intestinal inflammation, namely Chronic Inflammatory Bowel Diseases (CIBD), known internationally as Inflammatory Bowel Disease (IBD), in humans. In particular, a non-invasive method for assessing an intestinal inflammatory disease status in humans by detecting an increased level of the Rho GDP dissociation inhibitor 2 protein in fecal extracts is described.

### STATE OF THE ART

IBDs are a group of chronic inflammatory affections that electively affect the large intestine (ulcerative colitis, UC) or any part of the digestive tract, from the mouth to the anal region (Crohn's disease, CD). There are different types of inflammatory bowel diseases, such as ischemic and lymphocytic colitis, the so-called “indeterminate colitis”, but CD and UC represent the most common affections.

The hypothesis that chronic intestinal inflammation is the consequence of an exaggerated cell-mediated immune response to commensal bacteria in genetically predisposed hosts is now commonly accepted.

The immune system of the intestinal mucosa is able to “interpret” the local microenvironment and not react to the commensal bacterial population (“tolerance”), maintaining the ability to respond to pathogens. In genetically predisposed individuals, IBD develops by breaking the balance between mucosal immune responses and enteric bacteria.

Massive stimulation by bacterial antigens causes a numerical increase and the immunological activation of CD4+T lymphocytes. Therefore, traditionally, the pathogenesis of IBD is attributed to an exaggerated aggressiveness of the effector T cells and the proinflammatory cytokines, which exceed the usual control mechanisms.

More recently, a primary role in the induction of IBD by components of innate immunity has emerged, which plays a central role in the control of intestinal microbes and is based on the expression of receptors that recognize highly conserved microbial regions.

IBDs, which have a prevalence between 1 and 1.5 cases per 1000 individuals, while the incidence is 7-10 new cases/100,000 individuals, can occur at any age, more frequently in patients between 15 and 30 years old and in those between 50 and 70 years old. IBDs are more frequent in the countries of northern Europe, but in recent years there has been a consistent increase in their incidence in Mediterranean Europe as well, probably due to changes in eating habits. Also cases of IBD with pediatric onset are gradually increasing. 25% of new patients are younger than 20 years, cases with even earlier onset have been reported, even in the first years of life.

The range of the clinical manifestations of IBD is extremely variable, sometimes expressing in mild forms with few symptoms and sometimes with a very pronounced and heavy symptomatology for the patient. Sometimes such few symptoms do not allow an immediate recognition of the pathology. The main feature of IBD is the presence of a chronic inflammation of the intestinal mucosa having intermittent course and, especially if underestimated, or not correctly diagnosed, can lead to severe complications.

Generally, the patient alternates periods in which he does not accuse the symptoms of the disease (normal alve, absence of abdominal pain) to others in which the disease resumes, sometimes presenting in a more severe form. Over time, CD may exhibit complications such as narrowing of the lumen of the intestinal tract affected by inflammation and, in the most extreme cases, intestinal obstruction. Fistula formations or abscesses can then occur. In the case of the UC, complications such as colon dilatation and neoplasia can result.

The symptoms characterizing CD and UC are generally very different: in fact, in most cases CD is initially manifested with diarrhea and abdominal pains located especially in the lower right part of the abdomen, corresponding to the section of the intestine where more frequently the disease is localized. The UC instead always show up diarrhea and presence of blood and mucus in the stool, which is often associated with a feeling of incomplete evacuation and anemia. In the acute phases of both CD and the UC, states of general malaise may appear, such as weight loss, fatigue, loss of appetite, fever.

Given the non-specificity of symptoms and possible complications from IBD, it is clear that the correct diagnostic approach to typical symptomatology by the gastroenterologist is a problem as common as it is complex.

Until recently, in order to evaluate the inflammatory state of the intestinal mucosa and make a certain diagnosis of the disease, invasive tests such as ileocolonscopy and subsequent histological examination, were necessary. Recently, however, the use of non-invasive markers has found great development: among these, one of the most reliable and safe is the determination of fecal concentration of calprotectin, a protein mainly found in neutrophils which, in the presence of inflammatory processes of the intestine, is released into the intestinal lumen and therefore can be detected in the feces.

The use of calprotectin as a non-invasive marker of inflammation of the intestinal mucosa is not limited to the laboratory diagnosis phase of IBD, but it is also used in the follow-up and monitoring of specific patient therapy. Calprotectin is indeed a very useful aid for the differential diagnosis between organic and functional disease. Currently fecal calprotectin is considered a reference marker in the diagnosis and monitoring of intestinal inflammations, its use is recommended in the clinical practice guidelines.

In fact, the level of calprotectin in the feces of patients affected by IBD is generally very high. In subjects with Irritable Bowel Syndrome (IBS) the level of calprotectin is slightly above the normal limit and much lower than that found in patients with active disease, but always higher than that found in healthy subjects.

However, the literature data report that it works better in determining the UC than CD [Mao R. et al. Inflamm Bowel Dis 2012; 18: 1894-1899. Lin J. F. et al. Inflamm Bowel Dis 2014; 20: 1407-1415]. Furthermore, meta-analysis data showed that the patient's age affects the sensitivity of the diagnostic test because the marker works better in adults (sensitivity 93%, specificity 96%) than in pediatric patients (sensitivity 92%, specificity 76%) [van Rheenen P. F. et al. BMJ 2010; 341: c3369].

It has been also observed that the levels of the High Mobility Group Box 1 protein (HMGB1), normally present in the cell and released into the extracellular matrix during infections, cell stress or tissue damage, is significantly increases in the faeces of patients with IBD compared to healthy controls. Therefore, also the determination of HMGB1 in a patient's stool can be used as a valid marker of intestinal inflammation, as described in Italian patent no. 1406051, wherein it has been shown that patients with a moderate disease severity (PCDAI/PUCAI disease index <25/60), undergoing therapeutic treatment, show a reduced presence of HMGB1, compared to those with severe disease. Furthermore, the level of fecal HMGB1 correlates with the severity degree of disease and the level of fecal calprotectin (Palone F. et al. Inflamm Bowel Dis. 2016; 22: 2886-2893). Therefore, this protein, beside being a good marker of inflammation, is mainly a good indicator of response to therapy and therefore can be used as a prognostic marker. However, the limitation of the use of the HMGB1 marker consists in not having at present a diagnostic kit based on the immunological detection of the HMGB1 protein by ELISA, rather than by Western Blot, which is not very practical to be carried out in routine laboratory analysis.

Due to the growing need to identify non-invasive methods to detect gastrointestinal inflammations, increasingly sensitive and specific, but also rapid and economical, much attention is still being paid to the identification of new molecules that meet these characteristics. Therefore, the identification of new faecal indicators is an important target today, given the lack of markers able to fully meet all the requisites necessary to be used with satisfaction in the clinical practice, that is: easy to perform, reproducible, not expensive, and reflecting the activity of intestinal disease with high sensitivity and specificity. The faecal calprotectin itself, although being a good marker of intestinal inflammation, has nevertheless raised some perplexities about its capacity to predict relapse in patients with CD [Costa F. et al., Gut 2005; 54: 364-8], in its correlation levels with the pediatric disease activity index (PCDAI) and, according to someone, also in its specificity [Sidler M A. et al., Inflamm Bowel Dis 2008; 14: 359-66; Gisbert J P and McNicholl, Dig Liver Dis 2009: 41: 56-66].

However, although some good faecal markers are already present on the market, it should be kept in mind that none of them guarantees 100% sensitivity and specificity; therefore, having more markers for a pathology, or a group of similar pathologies, would allow significantly increase the reliability of these tools, both reducing the cases of false negative and positive results, and in enabling a more accurate diagnosis of the disease as well as to follow up the course, by monitoring the severity degree and the response to therapies.

The RHO GTPases family control a wide variety of cellular processes, including cell adhesion, migration and proliferation. At any time, only a small fraction of all RHO GTPases present in the cell are in the active state and are associated with cell membranes. In the cytosol proteins are maintained in an inactive state by association with inhibitors of dissociation of the guanine nucleotide specific for RHO (RHOGDIs), then Rho GDI forms a complex with Rho and maintains it inactive in the cytoplasm.

Despite the great diversity in the GTPase family of the RHO family, three are their dissociation inhibitors (RHOGDIs). RHOGDI1 is the most abundant and best characterized family member, it is ubiquitous and able to interact with different RHO GTPases. RHOGDI2 is highly expressed in hematopoietic cells [Lelias J. M. et al. PNAS USA 1993; 90: 1479-83; Scherle P. et al. PNAS USA 1993; 90: 7568-72;], but also in other tissues and tumor cells; the Rho GDP dissociation inhibitor 2 binds in vitro with different RHO GTPases, however many of these interactions have not been confirmed in vivo. RHOGDI3 is the most divergent group of the three proteins and has a specific amino-terminal extension that targets the Golgi complex and other cell membranes. Although they were originally considered passive regulators, more recent studies have shown that members of the Rho GDP dissociation inhibitor family actually have a key role in regulating RHO GTPases and that their function is far more complex.

As regards the possible application of the Rho GDP dissociation inhibitor 2 in the diagnostic field, it should be noted that in the international patent application WO 2010/088187 a method for the diagnosis of neonatal mammalian sepsis that provides for the determination of the protein level in a biologic fluid sample of a subject is described.

The application to the oncology field is based on the observation that changes in the expression levels of RHOGDI1 and RHOGDI2 have been associated with many types of cancer, but the changes vary according to the type of cancer [Harding M A et al. Eur J Cancer 2010; 46: 1252-9]. For example, the expression of RHOGDI1 is upregulated in colo-rectal and ovarian cancer, and in these cases high levels of expression correlate with increased invasiveness and resistance to chemotherapeutic agents [Jones M. B. et al. Proteomics. 2002. 2: 76-84; Zhao L. et al. J Proteome Res. 2008; 7: 3994-40003; Zhao L. et al. The FEBS Journal. 2010. 277: 4195-4204]. The RHOGDI2 expression levels are also significantly altered in a wide range of tumors [Harding M. A., Theodorescu D. Urologic oncology. 2007; 25: 401-6; Abiatari I. et al. Molecular cancer therapeutics. 2009; 8: 1494-1504]. The expression of Rho GDP dissociation inhibitor 2 is increased in pancreatic cancer and correlates with the increased invasiveness [Koide N. et al. Clin. Cancer Res. 2006; 12: 2419-2426]. The opposite occurs in kidney cancer, wherein the expression of RHOGDI2 is decreased in invasive tumor cells, and such decrease correlates with the decreased survival of the patient. In mammary tumor the RHOGDI2 expression pattern is rather complex, there is evidence that the protein expression is biphasic, increases in the early stages of progression and significantly decreases in the phase of metastasis [Hu L. D. et al. Oncology reports 2007; 17: 1383-1389]. U.S. Pat. No. 8,642,349 includes a tumor classification method useful for identifying the site of origin of a metastatic tumor in the event that no primary tumor is identified, entirely based on the quantified protein expression by two-dimensional gel electrophoresis (2DE). Thus, groups of genes were identified for each type or tumor origin site: ovaries, colon, kidney, breast, lung and stomach, obtaining a typical two-dimensional electrophoresis profile that has been used to compare that detected in patients. In the embodiment of the invention comprising among the various biomarkers the series of tumor biomarkers having a site of origin in the lung, the Rho GDP dissociation inhibitor 2 is also provided. From the studies conducted on RHOGDI proteins and their applications in the diagnostic field it clearly appears that their effects on diseases are complex and do not adapt to a simple explanatory model. It must be taken into account that the consequences of eventual changes in their expression become evident through their actions on multiple RHO GTPases, and that the levels and activities of these effectors vary significantly in the different cell types under physiological and pathological conditions.

The international patent application WO 2007/140508 teaches a method for detecting/diagnosing/determining ex vivo inflammatory conditions by evaluating an increase of a marker such as e.g. Rho GDP-Dissociation Inhibitor 2 in samples from a subject suffering from cystic fibrosis. The same document also teaches that: accordingly, a diagnostic and prognostic assay that determines the glycosylation patterns of mucins in a cystic fibrosis patient may be used as a general indicator of the degree of inflammation and/or infection in the respiratory tract or even the intestinal tract.

Shkoda A. et al. in “Differential protein expression profile in the intestinal epithelium from patients with inflammatory bowel disease”, Journal of Proteome Research. 2007, 6: 1114-1125) discloses differential expression of Rho GDP-dissociation inhibitor (GD1a) in patients with inflammatory diseases including ulcerative colitis (UC) and Crohn's disease (CD), however the publication does not assess the level of Rho GDP-Dissociation Inhibitor 2 (or beta).

### SUMMARY OF THE INVENTION

The present invention is based on the unexpected observation that the increased level of Rho GDP dissociation inhibitor 2 detected in the stool of a subject is associated with conditions of intestinal inflammation, in particular of CD and UC. The results obtained in the experimentation conducted by the Applicants have demonstrated that the detection of the faecal protein is not only useful to make a certain diagnosis of the condition, enabling to distinguish healthy from affected subjects, but it also provides a determination of the degree of severity of the condition, allowing to distinguish healthy subjects, subjects wherein the disease is inactive, and those suffering from mild or severe inflammation. In fact, the level of Rho GDP dissociation inhibitor 2 in stool correlates significantly with the current endoscopic indices used in clinical practice, Simple Endoscopic Score (SES-CD) for CD and the Mayo endoscopic index, normally used to express the degree of severity of the UC. Therefore, the detection of the protein is also useful as a prognostic indicator to follow the course of the disease and the possible response to a given treatment for intestinal inflammation.

A first aspect of the present invention is the use of the faecal Rho GDP dissociation inhibitor 2 in the diagnosis of inflammatory bowel disease, wherein in particular the inflammatory bowel disease is represented by CD and UC.

A second aspect of the present invention is the use of the Rho GDP dissociation inhibitor 2 in the prognosis of inflammatory bowel disease, wherein in particular the inflammatory bowel disease is represented by CD and UC.

In both diagnosis and prognosis, an increased level of the protein detected in the faeces of a subject is associated with the intestinal inflammation disease condition.

### BRIEF DESCRIPTION OF THE FIGURES

With reference to the attached figures, it should be noted that the expression “RGDI2” corresponds to the acronym of “Rho GDP Dissociation Inhibitor 2”.

FIG. 1 shows the graph that represents the concentration of the Rho GDP dissociation inhibitor 2 in the faecal sample of healthy subjects and of subjects suffering from Crohn's disease (A); in (B) the protein concentration is presented in the same affected subjects, subdivided according to the degree of severity of the disease (e.g. inactive, moderate and severe) assessed by endoscopy.

FIG. 2 shows the graphs which represents the concentration of Rho GDP dissociation inhibitor 2 in the faecal sample of healthy subjects and of subjects suffering from ulcerative colitis (A); in (B) the protein concentration is presented in the same affected subjects, subdivided according to the degree of disease severity (e.g. inactive, moderate and severe) evaluated by endoscopy.

FIG. 3 shows the graph presenting the correlation between the level of calprotectin and that of Rho GDP dissociation inhibitor 2 in the faecal sample of subjects affected by Crohn's disease.

FIG. 4 shows the graph showing the correlation between the level of calprotectin and that of Rho GDP dissociation inhibitor 2 in the faecal sample of subjects suffering from ulcerative colitis.

FIG. 5 shows the graph showing the correlation between the level of Rho GDP dissociation inhibitor 2 in subjects affected by Crohn's disease and the endoscopic index (SES-CD), normally used for this disease, which reflects the severity of the disease.

FIG. 6 shows the graph showing the correlation between the level of Rho GDP dissociation inhibitor 2 in subjects suffering from ulcerative colitis and the endoscopic index (Mayo), normally used to express the degree of severity of the disease.

### DETAILED DESCRIPTION OF THE INVENTION

The invention consists in using the level of Rho GDP dissociation inhibitor 2 present in a faecal sample of a subject as a diagnostic marker of the intestinal inflammatory pathologies and, therefore, in the determination of the level of such protein in the stool of a subject, and in its comparison to a predetermined value identified in the faeces of a group of subjects wherein a diagnosis of inflammatory bowel disease has been placed with clinical and laboratory techniques known to skilled in the field, such as the endoscopic investigation.

According to the invention the subject on which the level of Rho GDP dissociation inhibitor 2 is determined is a mammal, preferably it is a primate, even more preferably it is a human being. The value of the concentration of the Rho GDP dissociation inhibitor 2 can be obtained by any conventional technique suitable for qualitatively and quantitatively determining a protein present in a faecal sample, known to those skilled in the art, such as Western blot, two-dimensional electrophoresis, proteomics, chromatographic techniques; however, according to the invention, preferably the concentration of the Rho GDP dissociation inhibitor 2 is determined by immuno-absorbing assay bound to specific antibody (ELISA).

The value of concentration of the Rho GDP dissociation inhibitor 2 identified in the faeces of a group of healthy subjects, i.e. subjects that at the time of detection do not show signs of the disease and in which a diagnosis of inflammatory bowel disease posed with clinical and laboratory techniques known to skilled in the field has been ruled out, ranges from 0 to 42 pg/ml (average value 20 pg/ml), the value of 42 pg/ml is therefore the threshold value below which the subject is considered healthy, while above which the subject presents the disease.

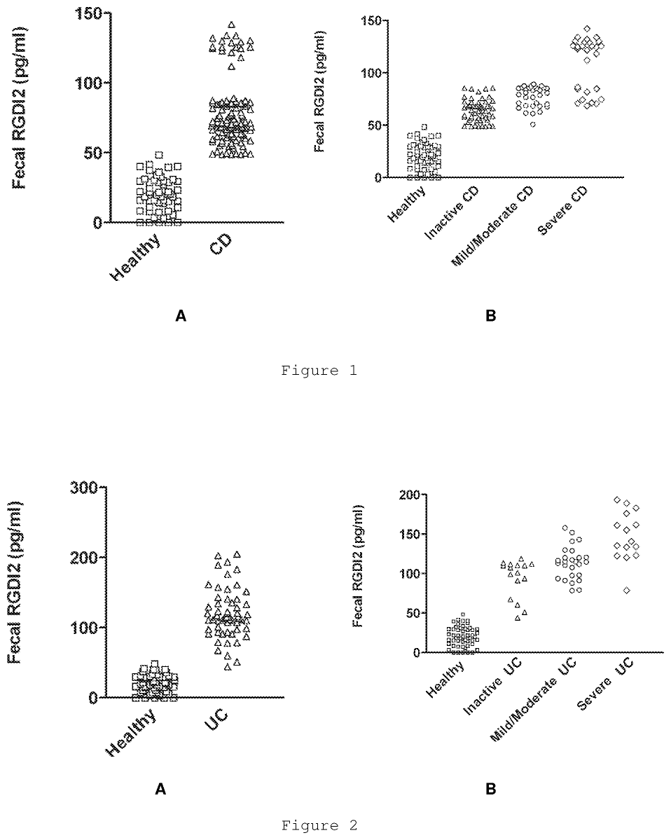

As shown in FIGS. 1 and 2, it was observed that in subjects suffering from inflammatory bowel disease, particularly in individuals with Crohn's disease and ulcerative colitis, the level of Rho GDP dissociation inhibitor 2 detected in a faecal sample is significantly higher than in healthy subjects.

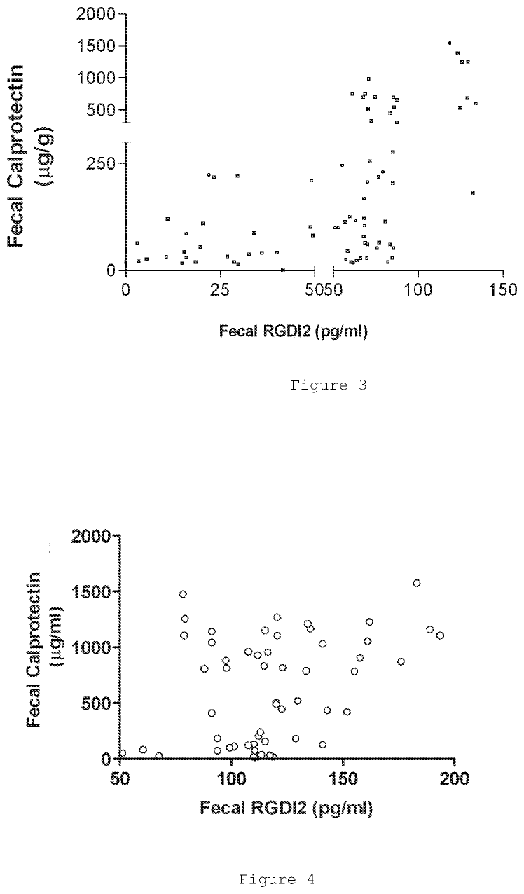

Furthermore, the concentration of Rho GDP dissociation inhibitor 2 in faeces finds excellent correlation with parameters obtained by other methods of diagnosis, both invasive and non-invasive. For example, a correlation was made between the faecal levels of Rho GDP dissociation inhibitor 2 with the levels of faecal calprotectin, currently considered the non-invasive diagnostic marker of choice for inflammatory bowel diseases. The comparison of faecal samples of affected individuals from both Crohn's disease and ulcerative colitis showed a positive and statistically significant correspondence between the two markers, as shown in FIGS. 3 and 4.

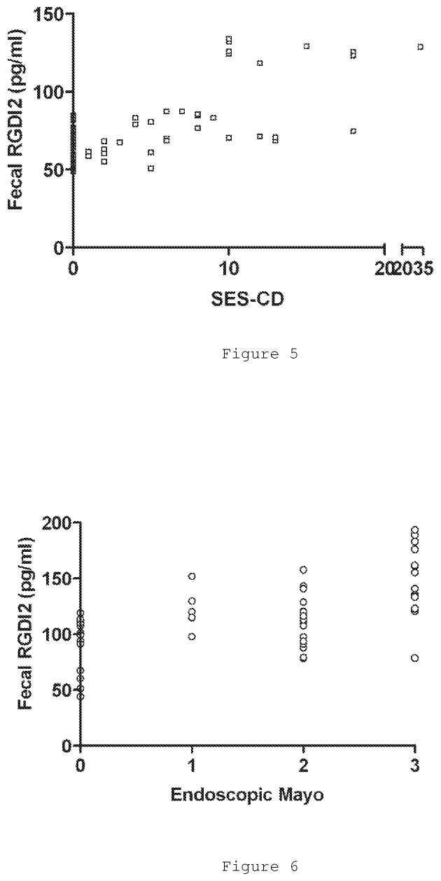

The correlation is maintained even when faecal levels of Rho GDP dissociation inhibitor 2 are compared with the endoscopic SES-CD index used for Crohn's disease, which classifies the degree of severity of the disease as a function of the size of ulcers present in the mucosa, the ulcerated surface, the extension of the surface of the affected mucosa and the presence of stenosis [Daperno M. et al., Gastrointest Endosc 2004; 60: 505-512. Moskovitz D. N., Disease. Gastroenterology 2007; 132: S1097]. Furthermore, SES-CD is related to clinical parameters and to the serum level of C-reactive protein. The correlation analysis data is shown in FIG. 5.

The correlation was also demonstrated for ulcerative colitis, in fact the correspondence between the endoscopic index Mayo Score, evaluating the stage of ulcerative colitis based on the four components: frequency of defecation, rectal bleeding, endoscopic evaluation and overall judgment [Schroeder K W et al., N Eng J Med 1987; 317: 1625-1629; Rutgeerts P. et al., N Engl J Med. 2005; 353: 2462-2476], and faecal levels of Rho GDP dissociation inhibitor 2 was observed. As shown in FIG. 6, there is a correspondence between the score attributed by the Mayo Score endoscopic index, which varies from zero (normal value or inactive pathology) to 3 (severe activity) and the protein concentration in the faeces.

The determination of the faecal concentration of the Rho GDP dissociation inhibitor 2, besides being useful as a diagnostic marker, has proved to be a valid prognostic tool, useful in assessing the real course of the disease and the response to therapy. In fact, when the disease enters remission, the level of protein returns to a physiological level, that is comparable with that of healthy subjects. Therefore, the fecal Rho GDP dissociation inhibitor 2 can be used not only to diagnose the condition, but also to confirm in the patient the actual state of remission of the disease, and, when an increase in protein values occurs, predict an imminent relapse and to assess whether and to what extent the patient responds to the therapy. Thus, the invention provides a method for determining whether a subject will benefit from continuing with therapy, also providing indications on the appropriateness of the dosage of the therapy in place, or by a change of therapy. The benefit typically results in a reduction in the level of protein concentration in the faecal sample that correlates with a reduction in signs, symptoms and typical manifestations of the disease, or in a more rapid recovery from such signs, including, for example, diarrhea and abdominal pain, presence of blood and mucus in the stool, which is often associated with anemia, weight loss, fatigue, loss of appetite and fever. It is therefore clear that this method has important implications for the treatment of patients and for the clinical development of new possible therapies, in fact to be able to determine whether a person will benefit by continuing with therapy or changing doses or changing the therapy itself is of absolute clinical relevance and allows to identify those patients that are most likely to respond to the chosen and undertaken therapeutic approach.

Actually, the experimentation has shown that the detection of the Rho GDP dissociation inhibitor 2 is also possible in the faeces of patients with disease defined as inactive on the basis of standard clinical indexes and which however still show a certain amount of intestinal inflammation, according to the evaluation based on the endoscopic score.

Thus, the present invention clearly demonstrates the usefulness and advantage deriving from the use of the Rho GDP dissociation inhibitor 2 detected in the fecal sample as a potential molecular prognostic parameter to predict possible recurrences in patients with IBD with apparent remission disease. In addition, due to the direct correlation between protein levels and disease severity, detection of the Rho GDP dissociation inhibitor 2 protein in faeces can be used as a prognostic marker of response to therapy. According to the invention, the determination of the level of Rho GDP dissociation inhibitor 2 is obtained by using conventional methods known to those skilled in the art, preferably obtained by an ELISA assay, or other immunological assays or conventional techniques for determining the presence of Rho GDP dissociation inhibitor 2, with appropriate modifications necessary to conduct the immunological assay on a biological sample as complex as the fecal matrix, which has very different characteristics compared to those of a body fluid such as saliva, urine, lymph, plasma, serum.

Currently, various kits comprising the reagents necessary for the determination of Rho GDP dissociation inhibitor 2 protein by a specific antibody-linked immuno-absorbent assay (ELISA) in a biological sample are commercially available. In a particularly preferred embodiment of the present invention for the determination of Rho GDP dissociation inhibitor 2 in the faecal sample, the SEE330Hu 96 Tests Enzyme-linked Immunosorbent Assay Kit for Rho GDP Dissociation Inhibitor Beta (ARHGDIb) product and commercialized by Cloud-Clone Corporation USA was used, but it will be evident to the skilled in the field that the choice of the kit is not limiting since any immuno-absorbent test linked to antibody specific for the Rho GDP dissociation inhibitor 2 protein can be used.

### CONCLUSION

The present invention has shown that the level, and in particular the increased level, of Rho GDP dissociation inhibitor 2 present in a faecal sample of a subject affected by inflammatory bowel pathology constitutes a reliable faecal diagnostic, prognostic and of response to therapies marker, able to correlate with the degree of severity of intestinal inflammation. The information obtained from the use of the protein as described is particularly valuable for clinicians who such way have more tools to monitor the condition of the patient and can, therefore, provide the most appropriate therapies.

Furthermore, the use of Rho GDP dissociation inhibitor protein as a faecal marker of intestinal inflammation allows to overcome the technical problems still present in the use of some diagnostic and prognostic markers, in particular the technical problems resulting from the use of other non-invasive diagnostic markers, such as faecal concentration of HMGB1.

In fact, compared to the use of the determination of the level of HMGB1, for which at present there is no specific antibody functioning in an ELISA test, the advantage resulting from the use of the Rho GDP dissociation inhibitor 2 is evident, since this determination can be carried out in a simple, rapid and reproducible manner by means of an ELISA assay in the analysis laboratory. Therefore, the introduction of the use of the determination of the faecal Rho GDP dissociation inhibitor 2 protein as a diagnostic and prognostic marker of intestinal inflammation does not simply represent a valid alternative to the diagnostic and prognostic methods of the state of the art, but contributes to a technological advancement overcoming technical problems still related to their use.

### Experimental Part

Preparation of the Faecal Sample

Fecal samples were obtained from 57 patients with Crohn's disease and 60 patients with ulcerative colitis (Table 1), with different level of disease severity, and 31 healthy controls, recruited at the Unit of Gastroenterology and Pediatric Hepatology of University “La Sapienza” of Rome-Policlinico Umberto I. Patients with an acute-severe form of the disease, suffering from toxic megacolon, subjects with complications requiring immediate surgery were excluded from the study. All the subjects who took part in the study were informed of the trial and gave their consent in writing. Samples, collected in sterile containers for faeces, were stored at −20° C. until molecular analysis.

TABLE 1
Characteristics of patients enrolled in the clinical study

| | n = 117 | | |
|---|---|---|---|
| | IBD Patient group | | |
| | | Crohn's | Ulcerose |
| | | Disease | Colitis |
| Patients | | n = 57 | n = 60 |

| | | | | |
|---|---|---|---|---|
| Age (mean, range) | 46 | (21-76) | 49 | (24-83) |

| | | |
|---|---|---|
| Gender (male/female) | 27/30 | 33/27 |

Disease features (n)

| | | |
|---|---|---|
| Inactive | 27 | 17 |
| Ulcerative colitis | — | 3 |
| Left Colitis | — | 3 |
| Extense Colitis | — | 37 |
| Ileo | 7 | — |
| Colonic | 6 | — |
| Ileocolonic | 16 | — |

| Inflammation localized in | 1 | (3%) | — | |
|---|---|---|---|---|
| the upper gastrointestinal | | | | |
| tract | | | | |
| Endoscopic activity of | | | | |
| the disease (n, %) | | | | |
| Inactive | 27 | (47%) | 17 | (28%) |
| Moderate | 16 | (28%) | 26 | (44%) |
| Severe | 14 | (25%) | 17 | (28%) |
| Endoscopic remission | | | | |
| Istologic Inflammation | 9 | (33%) | 5 | (29%) |
| Istologic remission | 18 | (67%) | 12 | (71%) |

Stool Weighing and Suspension in the Buffer

Each stool sample (about the size of an azelnut, equivalent to the contents hold by the little teaspoon inside a standard container for faeces) was taken with a sterile tip from the container, placed in a 1.5 ml eppendorf tube and weighed. The sample was resuspended in extraction buffer (salt phosphate buffer pH 7.2) containing detergent, Triton X-100, and sodium azide (ScheBo Biotech), to obtain a concentration of 500 mg/ml.

Stool Homogenization and Extraction

The sample was vigorously mixed with vortex for one minute at room temperature (RT); the sample was then placed in orbital excitement for an hour at RT. Then, it was mixed and centrifuged for 5 minutes at 5000 RPM at a temperature of 4° C. The supernatant was then taken and centrifugation was repeated. The supernatant called “faecal extract” was taken. At this point, the sample can be analyzed immediately by an ELISA test or stored at −80° C. and successively analyzed.

Analysis of Faecal Extracts by ELISA

All ELISA assays were performed using commercial kits following the instructions for use provided by the manufacturer.

The microplate provided in the kit used according to the invention is pre-coated with an antibody specific for the Rho GDP dissociation inhibitory 2 protein. The faecal samples were diluted to 1:50 using the dilution buffer provided in the kit. A volume of 100 μl of standard or sample is then added to the appropriate wells of the microtiter plate with a reagent containing the antibody specific for the Rho GDP dissociation inhibitory 2 protein conjugated with biotin. Subsequently, a second reagent containing avidin conjugated to horseradish peroxidase (HRP) is added to each well of the microplate and incubated. When the substrate solution, 3,3′,5,5′-tetramethylbenzidine or TMB is added, only in those wells containing Rho GDP dissociation inhibitory 2 protein, the biotin conjugated antibody and the avidin conjugate enzyme will show a color change. The reaction of the enzymatic substrate is terminated by addition of a sulfuric acid solution provided in the kit and the color change is spectrophotometrically measured at the wavelength of 450 nm±10 nm. The concentration of the Rho GDP dissociation inhibitor 2 in the samples is then determined by comparing the O.D. of the sample to the standard curve.

The reading of the O.D. in each sample it was conducted in duplicate.

Results

FIGS. 1-6 show the graphical representations of the data obtained in the experiment conducted to validate the use of the determination of the level of the Rho GDP dissociation inhibitory 2 in a faecal sample as a non-invasive diagnostic and prognostic marker, for intestinal inflammations.

FIG. 1 shows the data of the analysis performed by means of an ELISA test performed on faecal samples of patients with Crohn's disease and healthy controls. The results show that the Rho GDP dissociation inhibitory 2 in the faecal sample are significantly higher in patients compared to controls. Furthermore, when patients are divided according to disease severity evaluated by endoscopy, such as: inactive, moderate and severe disease, it is observed that the levels of the fecal Rho GDP dissociation inhibitory 2 protein increase significantly as the severity of the disease increases.

Subject healthy vs. CD: P<0.001 (***)

Subject healthy vs. inactive disease: P<0.001 (***)

Inactive disease vs. moderate: P<0.075 not significative.

Moderate disease vs. severe: P<0.05 (*)

FIG. 2 shows the data of the same analysis carried out on faecal samples of patients suffering from ulcerative colitis. Also in this case the analysis showed that the levels the fecal Rho GDP dissociation inhibitory 2 protein are significantly higher in patients affected than in controls. Furthermore, even in the case of ulcerative colitis, the levels of the fecal Rho GDP dissociation inhibitory 2 protein increase significantly with the increase in the severity of the pathology evaluated by endoscopy. Statistical analysis conducted by Mann Whytney test,
Subject healthy vs. UC *** P<0.001
Subject healthy vs. inactive disease *** P<0.001
Inactive disease vs. moderate disease ** P<0.01
Moderate disease vs. severe. ** P<0.01.

FIG. 3 shows the positive and statistically significant correlation between the fecal calprotectin level and the level of the Rho GDP dissociation inhibitory 2 in patients affected by Crohn's disease, fecal dissociation protein inhibitor 2 of the Rho GDP vs. Fecal calprotectin: Spearman r=0.61, p<0.001 (***).

FIG. 4 shows the data of the same analysis carried out on fecal samples of patients suffering from ulcerative colitis, fecal Rho GDP dissociation inhibitory 2 protein vs. fecal calprotectin: Spearman r=0.34, p<0.01 (***).

FIG. 5 shows the positive and statistically significant correlation between the level of Rho GDP dissociation inhibitory 2 in patients suffering from the Crohn's disease and the endoscopic index used for the classification of disease severity, fecal Rho GDP dissociation inhibitory 2 protein vs. SES-CD: Spearman r=0.64, p<0.001 (***).

FIG. 6 shows the correlation between the level of Rho GDP dissociation inhibitory 2 protein in patients with rectal ulcerative colitis and the endoscopic index used for the classification of disease severity, fecal Rho GDP dissociation inhibitory 2 protein vs. Mayo: Spearman r=0.63, p<0.001 (***).

1. A method for diagnosing/prognosing of intestinal inflammatory diseases by ex vivo detection of the level of human Rho GDP-Dissociation Inhibitor 2 protein in faecal samples of patients wherein an increased level of Rho GDP-dissociation inhibitor 2 detected in the faeces of the subject is associated with intestinal inflammation.

2. The method according to claim 1 wherein the concentration value of the human Rho GDP-Dissociation Inhibitor 2 protein can be obtained by a technique for qualitatively and quantitatively determining the protein in a faecal sample.

3. The method according to claim 2, wherein the technique for qualitatively and quantitatively determining the protein present in the faecal sample is selected from the group consisting of: two-dimensional electrophoresis, proteomics, chromatography, Western blot and immunoblotting and enzyme-linked immunosorbent assay (ELISA).

4. The method according to claim 1, wherein the concentration value of said protein in the faecal sample is detected by immunoblotting and enzyme-linked immunosorbent assay (ELISA).

5. The method according to claim 4, comprising the following steps:
(a) weighing the fecal sample and suspension in an extraction buffer to obtain a concentration of 500 mg/ml;
(b) homogenizing the sample, mixing and centrifuging for 5 minutes at 5000 RPM at a temperature of 4° C. twice;
(c) detection of human Rho GDP-Dissociation Inhibitor protein level in the faecal extract obtained in (b).

6. The method according to claim 5 wherein the extracting buffer used in (a) is a saline buffer pH 7.2 comprising Triton X-100 and sodium azide.

7. The method according to claim 5, wherein the determination of the protein level in the faecal extract is obtained by means of a specific antibody-specific immunosorbent assay (ELISA).

8. The method according to claim 1, wherein the concentration value of the Rho GDP-Dissociation Inhibitor protein in the faecal sample of a subject suffering from intestinal inflammatory diseases is higher than 42 pg/ml.

9. The method according to claim 1, wherein intestinal inflammatory disease is: Crohn's disease and ulcerative colitis.

10. The method according to claim 2, wherein the concentration value of said protein in the faecal sample is detected by immunoblotting and enzyme-linked immunosorbent assay (ELISA).

11. The method according to claim 3, wherein the concentration value of said protein in the faecal sample is detected by immunoblotting and enzyme-linked immunosorbent assay (ELISA).

12. The method according to claim 2, wherein the concentration value of the Rho GDP-Dissociation Inhibitor protein in the faecal sample of a subject suffering from intestinal inflammatory diseases is higher than 42 pg/ml.

13. The method according to claim 3, wherein the concentration value of the Rho GDP-Dissociation Inhibitor protein in the faecal sample of a subject suffering from intestinal inflammatory diseases is higher than 42 pg/ml.

14. The method according to claim 4, wherein the concentration value of the Rho GDP-Dissociation Inhibitor protein in the faecal sample of a subject suffering from intestinal inflammatory diseases is higher than 42 pg/ml.

15. The method according to claim 10, wherein the concentration value of the Rho GDP-Dissociation Inhibitor protein in the faecal sample of a subject suffering from intestinal inflammatory diseases is higher than 42 pg/ml.

16. The method according to claim 11, wherein the concentration value of the Rho GDP-Dissociation Inhibitor protein in the faecal sample of a subject suffering from intestinal inflammatory diseases is higher than 42 pg/ml.
